(19) United States
(12) Patent Application Publication
(10) Pub. No.: US 2008/0085273 A1
(43) Pub. Date: Apr. 10, 2008

(54) VEHICLE FOR THE TRANSPORT OF A CHOSEN MOLECULE TO A CELL

(75) Inventor: Marcel Herman Jozef Ruiters, Groningen (NL)

Correspondence Address: PEACOCK MYERS, P.C., 201 THIRD STREET, N.W., SUITE 1340, ALBUQUERQUE, NM, 87102, US

(73) Assignee: Synvolux IP B.V., Groningen (NL)

(21) Appl. No.: 11/778,210

(22) Filed: Jul. 16, 2007

Related U.S. Application Data
(63) Continuation-in-part of application No. 11/737,679, filed on Apr. 19, 2007.
Application No. 11/737,679 is a continuation-in-part of application No. PCT/NL05/00754, filed on Oct. 20, 2005.

(30) Foreign Application Priority Data
Oct. 21, 2004 (NL) 1027311
Nov. 4, 2004 (NL) 1027417
Nov. 10, 2004 (NL) 1027479

(51) Int. Cl. A61K 39/395; A61K 38/00

(52) U.S. Cl. 424/130.1; 514/2

(57) ABSTRACT

Vehicle for the transport of a chosen molecule to a cell, comprising a SAINT-molecule which is bound to the chosen molecule by means of an electrostatic interaction, in which the SAINT-molecule is coupled to the linker molecule and the linker molecule is coupled to the cell specific ligand and in which the SAINT-molecule is covalently bound to the linker molecule.

### CROSS-REFERENCE TO RELATED APPLICATIONS

This application is a continuation-in-part application of U.S. patent application Ser. No. 11/737,679 entitled “VEHICLE FOR THE TRANSPORT OF A CHOSEN MOLECULE TO A CELL”, filed Apr. 19, 2007, which is a continuation of Patent Cooperation Treaty (PCT) Serial No. PCT/NL2005/000754, entitled “VEHICLE FOR THE TRANSPORT OF A CHOSEN MOLECULE TO A CELL”, to Synvolux IP B.V., filed on Oct. 20, 2005, and the specifications and claims thereof are incorporated herein by reference.

This application claims priority to and the benefit of the filing of Netherlands Patent Application Serial No. 1027311, entitled “Vehicle to Transport a DNA-Modifying Enzyme to a Genome”, to Synvolux IP B.V., filed on Oct. 21, 2004, and the specification and claims thereof are incorporated herein by reference.

This application claims priority to and the benefit of the filing of Netherlands Patent Application Serial No. 1027417, entitled “Vehicle for the Transport of a Chosen Molecule to a Cell”, to Synvolux IP B.V., filed on Nov. 4, 2004, and the specification and claims thereof are incorporated herein by reference.

This application claims priority to and the benefit of the filing of Netherlands Patent Application Serial No. 1027479, entitled “Protection of Biologically Active Molecules Using Amphiphiles”, to Synvolux IP B.V., filed on Nov. 10, 2004, and the specification and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

The presented invention relates to a vehicle for the transport of a chosen molecule to a cell, comprising a SAINT-molecule, which by means of an electrostatic interaction, more particularly a non-covalent binding, for instance by hydrogen bonding, is bound to the chosen molecule. Further the invention relates to the application of the SAINT-molecule for a targeted transport of a chosen molecule to a cell.

### DESCRIPTION OF RELATED ART

It is known in the state of the art to administer chosen molecules to a body, in such a way that the chosen molecule has to perform its action in or near the preferred cell of the body. One possibility to deliver such molecules to a cell is the administration of considerable amounts of chosen molecules to the body, whereby, by diffusion, a required concentration of the chosen molecule will be obtained at the targeted cells. A problem of this process is that the chosen molecule might generate undesirable site effects in other parts of the body.

Therefore processes have been developed to deliver chosen molecules, for instance drugs, to a specific location in the body at a specific cell. A first method applied comprises the coupling of the chosen compound to antibodies. Such a coupling is covalent. As an effect, the activity of the chosen compound is substantially reduced in comparison with the activity of the free molecule. Another, currently applied method, is the packaging of the chosen molecules in liposomes. To the outside of the liposome, in which the molecule is packaged, an antibody can be coupled. This coupling of the antibody to the liposome occurs mostly after the inclusion of the chosen molecule in the liposome. In case the coupling of the antibody occurs before the chosen molecule has been included, it is possible that the antibody is located at the inner membrane of the liposome which might result in a decreased efficacy of the liposome. A general known disadvantage of inclusion of chosen molecules, for instance pharmaceutical compounds, in liposomes, is that pharmaceuticals will slowly leak from the liposome. A second disadvantage is that the formation of the packaging (the liposome containing the chosen compound inside) has to be performed in a solution containing the chosen compound. During packaging, not more than 5% of the chosen compounds will be included in the liposome. This means that 95% of the chosen compound is not included in the liposomes. Consequently, the efficacy of this method is very low.

### BRIEF SUMMARY OF THE INVENTION

The present invention aims at providing a vehicle which strongly improves the delivery of a chosen compound to a cell.

Furthermore the invention aims to provide a vehicle that enables long-lasting and stable binding to the chosen molecule.

To reach at least one of the aforementioned goals, the present invention provides a vehicle as mentioned in the preamble, characterized by the measures according to claim 1. In this way, a very stable process is provided to deliver a chosen molecule at a cell.

Although below hydrogen interaction and hydrogen bonding will be mentioned in general, the interaction is not limited to this single form. In all cases, any kind of electrostatic interaction is referred to.

### BRIEF DESCRIPTION OF THE SEVERAL VIEWS OF THE DRAWINGS

The accompanying drawing, which is incorporated into and form a part of the specification, illustrate one or more embodiments of the present invention and, together with the description, serve to explain the principles of the invention. The drawing is only for the purpose of illustrating one or more preferred embodiments of the invention and is not to be construed as limiting the invention. In the drawing:

FIG. 1 is the result of Human Embryonic Kidney cells, strain 293A, cultured in 12 well plates and are transfected with 4 combinations of SAINT-molecules (i.e. SAINT-18, SAINT-18 coupled to RGD, a mixture of SAINT-18 and SAINT-18 coupled to RGD, and SAINT-18 linker).

### DETAILED DESCRIPTION OF THE INVENTION

In this text the word “cell” is used in general. With this, however, as the person skilled in the art will clearly understand, a “specific cell” is mentioned towards which the chosen compound has to be transported to.

Preferred embodiments are described in claims 1, 2, and 3.

According to another aspect, the invention relates to the application of a SAINT-molecule to transport a chosen molecule, in a targeted way, to a cell. SAINT-molecules are commonly known, also described as a transport vehicle, and are extensively described in European patent publication number EP-0755924 B1 and in U.S. Pat. Nos. 5,853,694 and 6,726,894, which is registered to the present applicant. The description of these patent-applications is herewith included by reference, in the present application. SAINT-molecules are, according to a broad description, to be regarded as synthetic amphiphiles.

The way chosen molecules, for instance macromolecules or pharmaceuticals in general, are enwrapped in SAINT-molecules, is described extensively in the aforementioned European patent EP 0755924 BI1 and U.S. Pat. Nos. 5,853,694 and 6,726,894.

According to a preferred embodiment, the SAINT-molecule is covalently bound to a linker molecule. By means of this covalently coupling it is assured that is an integrated and solid part of the stable vehicle. The linker molecules might be bound to the SAINT-molecules at the positions R1, R2, R3, R4, R5 and R5′, taking into account that R5 and R5′ either can be identical or different. For a more extended description of these type of SAINT-molecules reference is made to U.S. Pat. No. 6,726,894. Examples of the linker molecules are for instance the following: 1-N′-Methyl-4-(aminobutyl, cis-9-oleyl)-methylpyrimidiniumchloride (SAINT-amino-linker), and 1-N′-methyl-4-(N-succinimidyl S-acetylthio-acetate, cis-9-oleyl)-methylpyridiniumchloride (SAINT-SATA-linker). In these both cases the acetylthioacetate and the SATA group function as the linker (moiety). However equivalent compounds also might be applied as linker-moiety, including every chemical compound which can be covalently bound to SAINT-molecules. Although in general only one linker molecule will be bound to the SAINT-molecule, it might be possible that a combination of positions is occupied by a linker molecule, for example R1, R2, R3, R4, R5 or R5′ alone or a combination of these, with a maximum off all five linker positions.

According to a further preferred embodiment, the cell specific ligand is chosen from: an antibody or a derivate thereof, a protein, a peptide, a compound with a specific application as a target for a cell surface, and other compounds with the desired cell specificity.

The antibodies or derivates thereof may be generated synthetically, or naturally occurring variants.

Within the scope of the present invention a ligand refers to every compound which is able to bind to a receptor or protein of a cell. A precondition for the ligand is that it has a specificity for a receptor and/or a cell type. Examples for these types of ligands are L-Dopa or adrenaline derivates. This first type of ligands specifically binds the dopamine receptors in the substantia nigra; the second type of ligand specifically binds to the adrenergic receptors of the body. Of course, the invention is not restricted to this example.

More specifically, it is preferred that the molecule to be enwrapped binds at the active surface of the SAINT-molecule. Without to be restricted to this embodiment, here the positively charged pyridinium group is used. This group binds to the negative charge of the macromolecule to be enwrapped. Furthermore it is possible that the Saint-molecules will bind the macromolecule in an indirect manner, by interacting with the water-layer surrounding the macromolecule, in case the macromolecule has a positive charge. Consequently, the SAINT-molecules will bind the macromolecules by means of an electrostatic interaction, for instance by forming hydrogen-bonds, so preventing leakage of the chosen molecules from the SAINT-molecules.

Furthermore the possibility exists that electrostatic interaction from, for instance, the carbon-atoms or the ortho-electrons of the pyridinium-ring, is for binding.

The present invention creates the possibility to deliver chosen molecules, for example a drug, at a specific place in the body at a specific cell. Hereby the advantage is generated that the amount of drugs, which has to been administered to the body, can be geared exactly for the amount of cells which needs to be treated. By choosing the appropriate formulation between the Saint-molecules and the enwrapped compound, leakage of the compound is prevented, and it is released after delivery to, fusion with, or uptake into the cell membrane, which also contains the receptor ligand that is complementary for the linker coupled ligand. In this way it is prevented that the chosen molecule is released at other places in the body. Here they stay included in the SAINT-molecule-vehicle.

The essence of the invention is described above. Based on the description above and the attached conclusions, a person skilled in the art will be able to simply develop further embodiments, which all will fall within the scope of the present invention.

### EXAMPLE

### Synthesis of 4-[1-(4-Amino-butyl)-nonadec-10-enyl]-1-methyl-pyridinium chloride (SAINT-amino-linker)

The synthesis of SAINT-amino-linker is performed according to scheme 1:

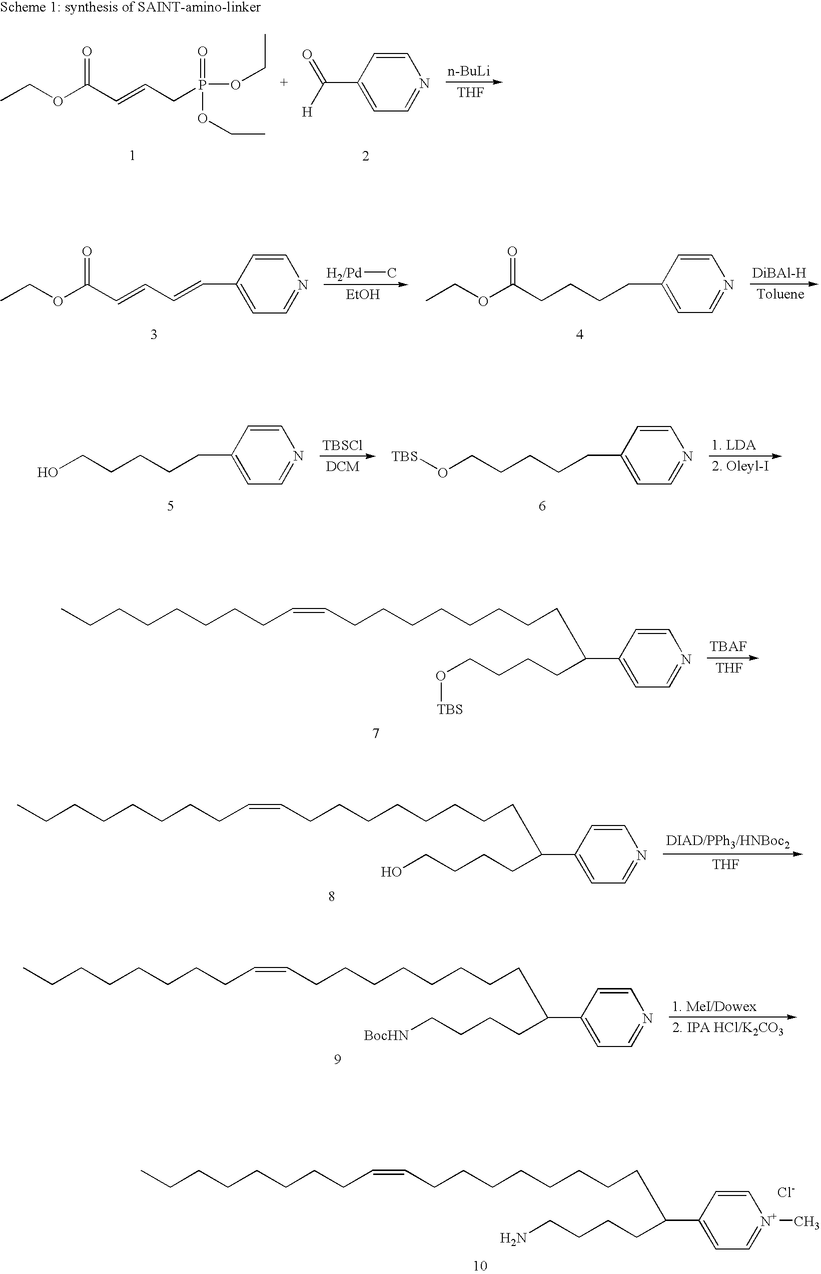

5-Pyridin-4-yl-penta-2,4-dienoic acid ethyl ester (3). To 250 mL tetrahydrofuran at −70° C. was added 32 mL n-butyl lithium (2.5 M in hexanes) and 11.25 mL (80 mmol) diisopropylamine. After 15 minutes of stirring 17.8 mL (80 mmol) of phosphonocrotonate was added. Another 30 minutes of stirring were applied and next 7.6 mL (80 mmol) of 4-pyridinecarboxaldehyde was added dropwise. The mixture was allowed to heat up to room temperature. The clear brown solution turned first into a yellow suspension, then in a brown suspension and was stirred overnight. The reaction was quenched with acetic acid and the solvents were evaporated. The resulting brown oil was neutralized with NaHCO₃ (saturated solution in water) and extracted twice with ethyl acetate. The combined organic layers were washed with brine, dried (Na₂SO₄) and evaporated. The remaining brown oil was dissolved in ethyl acetate, filtered over silica and evaporated. The remaining orange oil, 16.7 grams (82.3 mmol, >100%) contained some impurities but was used without further purification.

¹H NMR (200 MHz, CDCl3) δ 9.00 (2H, d), 7.90-7.60 (1H, m), 7.70 (2H, d), 7.50-7.30 (1H, m), 7.19 (1H, d), 6.45 (1H, d), 4.60 (2H, q), 1.65 (3H, t) ppm.

5-Pyridin-4-yl-pentanoic acid ethyl ester (4). 5-Pyridin-4-yl-penta-2,4-dienoic acid ethyl ester (16.7 g, 82.3 mmol) was dissolved in 500 mL ethanol. After the addition of 3.0 grams palladium on carbon the mixture was exposed to hydrogen pressure (1 bar) for 24 hours. The mixture was filtered over Celite and the yellowish filtrate was evaporated. The isolated product was a yellow oil 14.5 grams (70.0 mmol, 85%).

¹H NMR (300 MHz, CDCl₃) δ 8.45 (2H, d), 7.10 (2H, d), 4.05 (2H, q), 2.60 (2H, t), 2.25 (2H, t), 1.65-1.55 (4H, m), 1.20 (3H, t) ppm.

5-Pyridin-4-yl-pentan-1-ol (5). 5-Pyridin-4-yl-pentanoic acid ethyl ester (14.5 g, 70 mmol) was dissolved in toluene (250 mL) in a flame-dried 1 Liter flask under nitrogen atmosphere. The solution was cooled to −60° C. and DiBAl-H (250 mL, 1 M in toluene) was added dropwise. The mixture was stirred for 2 hours at −40° C. and was allowed to warm up to room temperature. Next methanol (250 mL) was added to the reaction. The mixture was stirred for 30 minutes and changed into a yellow suspension. The mixture was filtered over Celite, dried (Na₂SO₄) and evaporated: 6.2 g brown oil (37.5 mmol, 54%).

¹H NMR (300 MHz, CDCl3) δ 8.45 (2H, d), 7.18 (2H, d), 3.65 (2H, t), 2.60 (2H, t), 1.70-1.25 (6H, m) ppm.

4-[5-(t-Butyl-dimethyl-silanyloxy)-pentyl]-pyridine (6). 5-Pyridin-4-yl-pentan-1-ol (3 g, 18.2 mmol) was dissolved in 100 mL DCM and triethylamine (2.80 mL, 19.9 mmol) was added. After 10 minutes of stirring t-butyldimethylsilyl chloride (3.0 grams, 19.9 mmol) was added and the mixture was stirred at room temperature overnight. A volume-equivalent of water was added and the mixture was stirred for 5 minutes. The organic layer was separated and dried (Na₂SO₄). After evaporation of the solvent 5 grams of crude 6 was isolated as a brown liquid. Column chromatography with heptane:ethylacetate (5:1, R_{fprod}=0.22) yielded 2.5 grams pure 6 (49%).

¹H NMR (300 MHz, CDCl3) δ 8.45 (2H, d), 7.08 (2H, d), 3.60 (2H, t), 2.60 (2H, t), 1.70-1.30 (6H, m), 0.93 (9H, s), 0.01 (6H, s) ppm.

4-{1-[4-(t-Butyl-dimethyl-silanyloxy)-butyl]-nonadec-10-enyl}-pyridine (7). Tetra-hydrofuran (200 mL) was cooled to −17° C. under a nitrogen-atmosphere. n-Butyllithium (8.6 mL, 2.5 M in hexanes) was added and after 5 minutes of stirring diisopropylamine (3.0 mL, 21.5 mmol) and hexamethyl phosphoric acid triamide (3.75 mL, 21.5 mmol) were added. After 30 minutes of stirring 4-[5-(t-butyl-dimethyl-silanyloxy)-pentyl]-pyridine (2.0 g, 7.2 mmol) was added. After another 30 minutes of stirring oleyl iodide (3.0 g, 7.94 mmol) was added and the mixture was allowed to heat up to room temperature and was stirred overnight. The mixture was diluted with t-butylmethylether and washed twice with ammonium chloride (saturated solution in water) and once with brine. After drying (Na₂SO₄) and concentration in vacuo 3.7 grams of the crude product was isolated as a yellow oil. Purification by column chromatography (heptane:ethylacetate 5:1) yielded 1.6 of the pure product (42%). Oleyl iodide pas prepared from oleyl alcohol according to a procedure described in ‘The synthesis of 1-methyl-4-(1-octadec-9-enyl-nonadec-10-enyl)-pyridinium chloride’.

¹H NMR (300 MHz, CDCl₃) δ 8.45 (2H, d), 7.02 (2H, d), 5.38 (2H, t), 3.60 (2H, t), 2.45 (1H, m), 2.05-1.95 (4H, m), 1.70-1.30 (6H, m), 1.20-1.00 (22H, m), 0.98-0.90 (15H, s), 0.01 (6H, s) ppm.

5-Pyridin-4-yl-tricos-4-en-1-ol (8). 4-{1-[4-(t-Butyl-dimethyl-silanyloxy)-butyl]-nonadec-10-enyl}-pyridine (1.6 g, 3.0 mmol) was dissolved in 5 mL of tetrahydrofuran. Tetrabutylammonium fluoride (2.0 g, 6.3 mmol) was added and the mixture was stirred overnight at room temperature. When TLC (heptane:ethylacetate 1:1 R_{fprod}=0.22, R_{fsm}=0.78) showed complete conversion, one volume-equivalent of water was added. The mixture was extracted with t-butylmethylether. The organic layer was dried (Na₂SO₄) and evaporated, yielding 1.1 g (2.7 mmol) of the product as a yellow oil (88%).

¹H NMR (300 MHz, CDCl₃) δ 8.45 (2H, d), 7.02 (2H, d), 5.38 (2H, t), 3.60 (2H, t), 2.45 (1H, m) 2.05-1.95 (4H, m), 1.70-1.30 (6H, m), 1.20-1.00 (22H, m), 0.98-0.95 (3H, t) ppm.

(5-Pyridin-4-yl-tricos-14-enyl)-carbamic acid t-butyl ester (9). To a solution of 5-pyridin-4-yl-tricos-14-en-1-ol (415 mg, 1 mmol) in tetrahydrofuran (20 mL) at room temperature was added di-t-butyl iminodicarboxylate (1.5 mmol, 325 mg), triphenylphosphine (1.5 mmol, 393 mg) and diethylazodicarboxylate (1.5 mmol, 261 mg) and the mixture was stirred overnight. Column chromatography with heptane:ethylacetate (1:1, R_{fprod}=0.3, R_{fsm}=0.14) yielded 200 mg (0.39 mmol, 39%) of the mono-Boc-protected amine (9) as a colorless oil.

¹H NMR (300 MHz, CD₃OD) δ 8.45 (2H, d), 7.02 (2H, d), 5.38 (2H, t), 3.55 (2H, t), 2.45 (1H, m) 2.05-1.95 (4H, m), 1.70-1.00 (37H, m), 0.98-0.95 (3H, t) ppm.

4-[1-(4-Amino-butyl)-nonadec-10-enyl]-1-methyl-pyridinium chloride (10). 1.6 g (3.1 mmol) of (5-pyridin-4-yl-tricos-14-enyl)-carbamic acid t-butyl ester was dissolved in iodomethane (10 mL) and stirred for two hours. The iodomethane was removed in vacuo and the remaining yellow oil was dissolved in methanol and eluted over a Dowex column. After evaporation of the solvent 1.5 grams product remained (2.8 mmol, 91% over 2 steps).

¹H NMR (300 MHz, CDCl₃) δ 9.40 (2H, d), 8.70 (2H, d), 6.72 (1H, bs), 5.38 (2H, t), 4.70 (3H, s), 3.45 (2H, t), 2.79 (1H, m) 2.05-1.95 (4H, m), 1.90-1.00 (37H, m), 0.98-0.95 (3H, t) ppm.

400 mg (0.9 mmol) of 4-[1-(4-t-butoxycarbonylamino-butyl)-nonadec-10-enyl]-1-methyl-pyridinium chloride was dissolved in a solution of 5-6 M of HCl in i-propanol (25 mL) and stirred for 1 hour at room temperature. Evaporation of the solvent yielded the HCl-salt of the deprotected amine. The residue was dissolved in methylene dichloride and 0.5 g of potassium carbonate was added and the suspension was stirred for 1 hour at room temperature. After filtration and removal of the solvent a white foam remained (130 mg, 0.3 mmol, 40% over 2 steps).

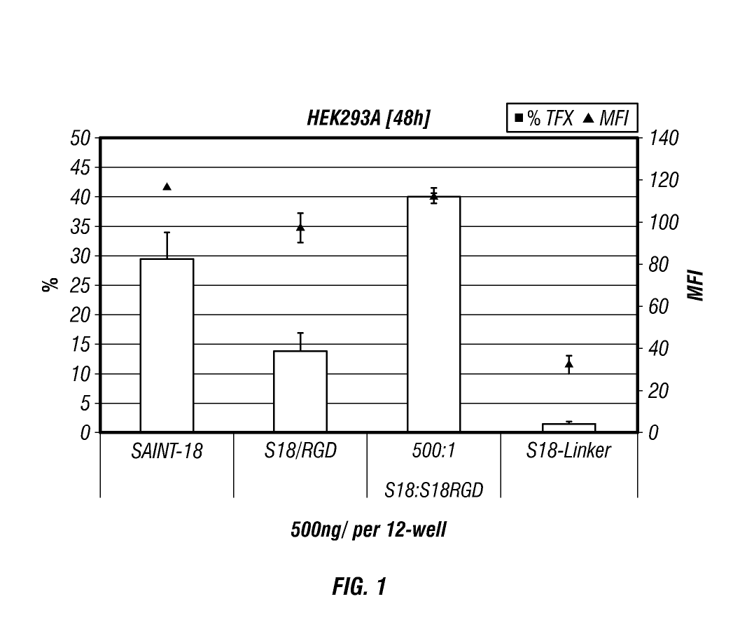

Human Embryonic Kidney cells are difficult to be transfected. When using these cells, main competing transfection methods (of other manufacturers), require several micrograms of plasmid DNA for successful transfection. Combined with SAINT-18, 1 μg (per 12 wells) normally results in a transfection rate above 90%. To be able to visualize the enhancing effect of the targeting moiety SAINT-18-RGD, in this protocol we used a low amount of plasmid DNA (500 ng per 12 wells). The structural formula of SAINT-linker-RGD is depicted in FIG. 1. In addition, an RGD-peptide is coupled to the SATA-group of the SAINT-Linker, as described in the present invention. The RGD group is a targeting moiety for the integrin receptor family and is though to increase the transfection efficacy. Human Embryonic Kidney cells, strain 293A, are cultured in 12 well plates and are transfected with 4 combinations of Saint-molecules (i.e. SAINT-18, SAINT-18 coupled to RGD, a mixture of SAINT-18 and SAINT-18 coupled to RGD, and only a linker molecule as a reference). 500 ng of CMV-GFP plasmid is complex with 3.75 nm SAINT molecules or linker (per well). 48 hours after transfection GFP expression is measured by FACS analysis. The percentage of GFP positive cells in depicted in the graph below.

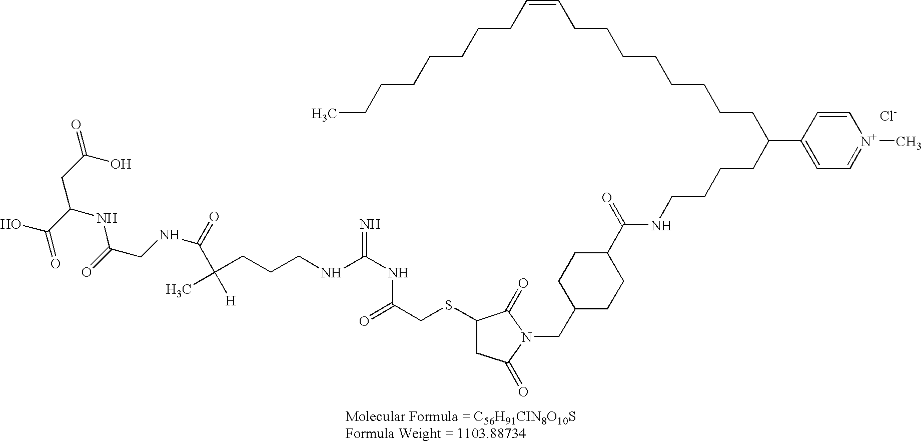

### Structural Formula of SAINT-Linker-RGD

The results depicted in FIG. 1 clearly show that

1. S18-linker (reference) functions poorly as a transfection reagent (column 4).

2. S18/RGD is able to transfect cells.

3. S18:S18RGD ratio 500:1 increases the transfection rate by approximately 33%.

S18-linker alone has no major transfection ability. It is anticipated that 100% S18-RGD has a decreased transfection efficacy resulting from the sterical hindrance generated by the RGD group. However when S18RGD is mixed with S18 in a ratio of 500:1, transfection efficacy is greatly increased. A more optimized ratio will be determined. Similar results are obtained with targeting moieties other than RGD (such as a cell-specific ligand). However, in all cases, an optimization will be needed to gain an optimal effect. This experiment greatly indicates the transfection enhancing characteristic of SAINT-molecules.

What is claimed is:

1. A vehicle for the transport of a chosen molecule to a cell, comprising a lipid, amphiphile or other type of drug delivery embodiment, which is mixed with SAINT-linker molecules, wherein the SAINT-molecule is covalently bound to the linker molecule which is covalently bound to one or more of the items selected from the group comprising of an antibody or a derivate thereof, a protein, a peptide, a compound with the specific application that it targets a cell-surface, and other compounds with desired cell specificity.

2. The application of a SAINT-molecule for the targeted transport of a chosen molecule to a cell, in which the SAINT-molecule is bound to the chosen molecule by means of an electrostatic interaction, and in which the SAINT-molecule is coupled to the linker molecule and the linker molecule is coupled to the cell specific ligand.

3. The application according to claim 2, using a vehicle according to the claim 1.
